(19) United States
(12) Patent Application Publication
(10) Pub. No.: US 2026/0060522 A1
(43) Pub. Date: Mar. 5, 2026

(54) DIGITAL BINOCULARS WITH DECENTERED MICRO-DISPLAY SCREENS FOR IMPROVED CONVERGENCE

(71) Applicant: Alcon Inc., Fribourg (CH)

(72) Inventors: Mauricio Jochinsen, Laguna Beach, CA (US); Vince Larocca, Santa Barbara, CA (US); Yuchao Liu, Irvine, CA (US); Yu Yan, Irvine, CA (US); Jing Xu, Irvine, CA (US)

(21) Appl. No.: 19/300,062

(22) Filed: Aug. 14, 2025

Related U.S. Application Data
(60) Provisional application No. 63/690,926, filed on Sep. 5, 2024.

(51) Int. Cl. A61B 3/00; A61B 3/13

(52) U.S. Cl. CPC A61B 3/005; A61B 3/132

(57) ABSTRACT

A digital ocular system for use with image sensors and an ophthalmic microscope includes a housing that connects to the microscope and defines a housing cavity, a first lens having a first optical axis, and a second lens having a second optical axis parallel to the first optical axis. A first micro-display is positioned within the housing cavity to present a first image from the image sensors. The first micro-display has a first center axis arranged at a predetermined angle with respect to the first optical axis. A second micro-display presents a second image from the image sensors. The second micro-display includes a second center axis arranged at the predetermined angle with respect to the second optical axis, such that the first and second micro-displays are horizontally decentered relative to the first and second optical axes, respectively.

### INTRODUCTION

The present disclosure relates to optimization of a visualization system equipped with digital oculars. In particular, the present solutions are directed to creating convergence when using the digital oculars to view target object, e.g., an eye in a representative ophthalmic application, for a more pleasant and natural viewing experience when using the digital oculars.

Digital and hybrid (i.e., analog/digital) camera-equipped microscopes are able to acquire digital images of a target object using image sensors. The acquired digital images may be magnified or reduced in size as needed depending on the field-of-view and the size of the image sensors. An ophthalmic microscope in particular includes three major components: (i) imaging optics providing two slightly different views to approximate normal human stereo vision, (ii) a pair of image sensors, e.g., complementary metal-oxide-semiconductor (CMOS) or charge-coupled device (CCD) sensors, along with a corresponding image processing unit for converting the collected image data to digital pixel images, and (iii) a high-resolution display screen for presenting the digital images to an attending clinician.

A head-up display (HUD) system may be used for the latter purpose in an ophthalmic surgical suite. In a typical HUD system, the clinician wears polarized glasses and views projected three-dimensional (3D) images on one or more display screens. While such a system provides high-resolution views of the patient's eye area, the need for polarized glasses and viewing of 3D images on the display screen(s) may be suboptimal for certain users or applications, for instance in terms of relative clinician comfort and image display qualities. Digital binoculars provide a possible in-suite viewing alternative to HUD systems, microscope ocular pieces, and other approaches. However, the projected images as presented to a user wearing digital binoculars can be difficult for some users to perceive from a physiological standpoint.

### SUMMARY

Disclosed herein is a visualization system having a microscope equipped with digital binoculars. The visualization system may be used as an alternative to a head-up display (HUD) system in an exemplary ophthalmic surgical suite to provide a more natural and comfortable view of a patient's eye. When using digital binoculars with an ophthalmic digital or hybrid microscope, where an image of a patient's eye is digitally acquired by a camera or other image sensors, the acquired images are displayed on two laterally-spaced miniature display screens or “micro-displays”, with one image presented to each eye of the viewing clinician. A binocular housing is mechanically coupled to the microscope, with the digital binoculars including eyepieces with front lenses having a particular eyepiece magnification level. The micro-displays contained within the housing are viewed through the eyepieces, which respectively correspond to a clinician's left and right eye. Each front lens is positioned relative to a respective one of the micro-displays contained within the aforementioned housing.

As described below, the micro-displays are horizontally decentered relative to a corresponding optical axis of each of the front lenses to help address a convergence problem commonly associated with viewing target objects using digital binoculars. Physiological discomfort in the form of fatigue, disorientation, or nausea may result when a projected image of a target object imperfectly matches the clinician's expected “real world” view of the target object, in this case a patient's eye. To address this concern and render digital binoculars fit for widespread use in an ophthalmic suite, the micro-displays are slightly horizontally decentered, e.g., by up to about 10% of a side length of the micro-display, to provide a fixed amount of optical decentering. In some embodiments, such optical decentering may be enhanced using digital decentering to provide the desired amount of convergence.

In a possible embodiment, a digital ocular system for use with image sensors and an ophthalmic microscope includes digital binoculars having a binocular housing configured to connect to the microscope. First and second front lenses of the binoculars have a respective first and second optical axis. Additionally, a first micro-display is positioned within the housing cavity and configured to present a first image from the image sensors, with the first micro-display having a first center axis arranged at a predetermined angle with respect to the first optical axis. Similarly, a second micro-display is positioned within the housing cavity and configured to present a second image from the image sensors. The second micro-display includes a second center axis arranged at the predetermined angle with respect to the second optical axis, such that the first and second micro-displays are horizontally decentered relative to the first and second optical axis, respectively.

Also disclosed herein is a visualization system having a microscope, a pair of image sensors connected to the microscope, and the digital ocular system summarized above.

In accordance with an exemplary embodiment, the first micro-display positioned within the housing cavity may have a square perimeter, with a side length of the square perimeter being about 20 mm-25 mm, e.g., 21 mm. The first micro-display is configured to present a first image from the image sensors. The first micro-display may have a first center axis arranged at an angle of less than about 2-degrees (°) with respect to the first optical axis. The first and second center axes are respectively offset from the first and second optical axes by about 0.5 mm to about 1.5 mm in this particular non-limiting embodiment.

The second micro-display may likewise have a side length of about 20 mm-25 mm, and may be configured to present a second image from the image sensors. As with the first micro-display, the second micro-display in this representative construction has a second center axis arranged at the angle of less than about 2° with respect to the second optical axis, such that the first micro-display and the second micro-display are horizontally decentered relative to the first optical axis and the second optical axis, respectively.

The above-described features and advantages and other possible features and advantages of the present disclosure will be apparent from the following detailed description when taken in connection with the accompanying drawings.

### BRIEF DESCRIPTION OF THE DRAWINGS

FIG. 1 illustrates an exemplary visualization system equipped with an ophthalmic microscope and digital binoculars having decentered micro-displays providing a slight amount of convergence to projected images in accordance with the present disclosure.

FIG. 2 illustrates representative digital binoculars having decentered micro-displays.

FIG. 3 is a schematic illustration of a micro-display that is horizontally offset from an optical axis of an eyepiece and front lens of the digital binoculars of FIG. 1.

FIG. 4 is a schematic illustration of horizontal decentering of a micro-display relative to an eyepiece/lens of the digital binoculars shown in FIG. 1.

The solutions of the present disclosure may be modified or presented in alternative forms. Representative embodiments are shown by way of example in the drawings and described in detail below. However, inventive aspects of this disclosure are not limited to the disclosed embodiments. Rather, the present disclosure is intended to cover alternatives falling within the scope of the disclosure as defined by the appended claims.

### DETAILED DESCRIPTION

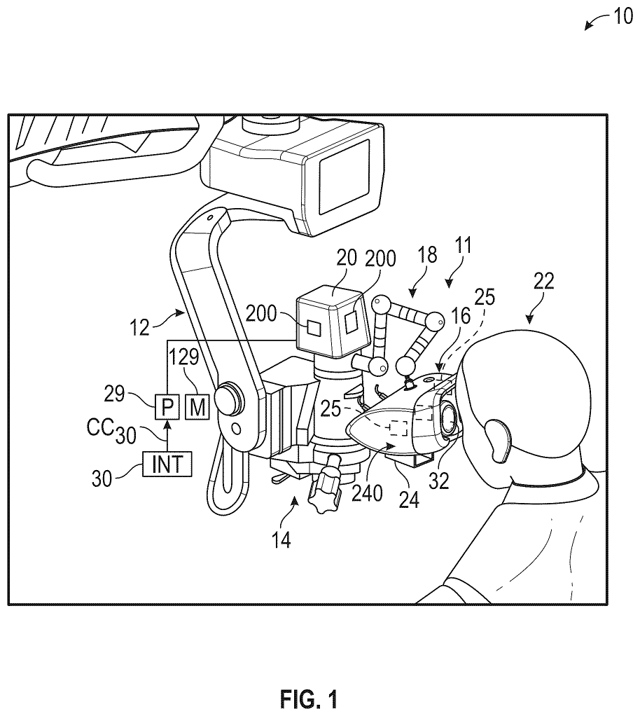

Referring to the drawings, wherein like reference numbers refer to like components, and beginning with FIG. 1, a visualization system 10 having a digital ocular system 11 is constructed in accordance with the present disclosure includes a mounting bracket 12 such as a C-mount, an ophthalmic microscope 14 coupled to the mounting bracket 12, and digital binoculars 16. The digital binoculars 16 include one eyepiece 32 per eye of a viewing clinician 22, with one eyepiece 32 being visible in FIG. 1. The digital binoculars 16 also include a binocular housing 24 defining a housing cavity 240. The housing 24 surrounds and protects various internal components of the digital binoculars 16 as set forth herein, including a pair of miniature display screens (“micro-displays”) 25 for viewing displayed images of a patient's eye (not shown) or another target object or anatomy. The housing 24 is shown in a non-limiting exemplary configuration, with other possible shapes, sizes, materials of construction, etc., being usable in other implementations.

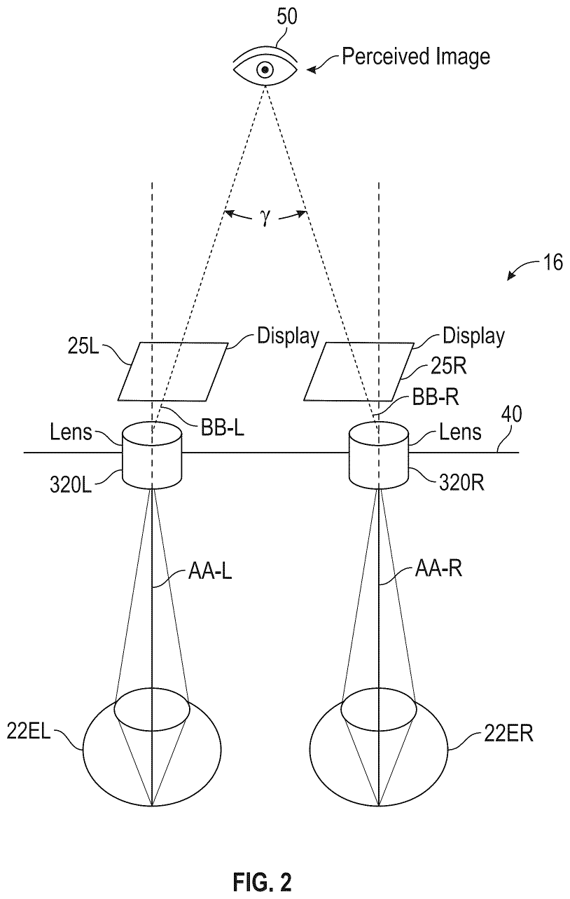

The eyepieces 32 include or are integrally formed with left and right front lenses 320L and 320R (see FIG. 2), with the suffixes “L” and “R” corresponding to the respective left and right eye of the clinician 22. Each front lens 320L and 320R has a corresponding eyepiece magnification level and object field-of-view, and is constructed with foreknowledge of the total magnification level of the visualization system 10 as a whole, as appreciated in the art. As contemplated herein, the digital ocular system 11 may include at least the binocular housing 24, the eyepieces 32 and their corresponding front lenses 32L and 320R, and the micro-displays 25.

In the representative embodiment of FIG. 1, the binocular housing 24 is mechanically coupled or connected to the microscope 14 via an articulated bracket 18. A digital camera 20 having a pair of image sensors 200 may be connected to the microscope 14, with the image sensors 200 variously embodied as, e.g., charge-coupled devices (CCDs), complementary metal-oxide-semiconductor (CMOS) sensors, electron-multiplying CCDs (EMCCDs), or another set of application-suitable digital image sensors. The image sensors 200 are configured to output high-resolution three-dimensional (3D) image data to the digital binoculars 16 for real-time viewing by the clinician 22, for instance a surgeon or attending medical staff working within an ophthalmic surgical suite. Each of the image sensors 200 collects a corresponding pixel image such that the clinician 22 views one image per eye on a corresponding one of the micro-displays 25 of FIG. 1.

Emerging digital binoculars based on image projection of virtual reality (VR) headsets come equipped with the eyepieces 32 to provide the digital binoculars 16 with a predetermined magnification level. The micro-displays 25 as described below with reference to FIGS. 2-4 are horizontally decentered or linearly offset to minimize vergence-accommodation conflict (VAC) when the clinician 22 views a projected image of a target object through the eyepieces 32. A processor (P) 29 and a user interface device (INT) 30, the latter outputting a control signal CC₃₀ as described below, may be used in some embodiments to provide supplemental digital decentering to thereby enhance the desired convergence effects of optical decentering as contemplated herein.

The various processes described herein may be embodied as computer-readable instructions and executed from a non-transitory computer readable storage medium/memory 129, for instance magnetic or optical media, CD-ROM, solid-state/semiconductor memory, and the like. The processor 29 may entail combinations of Application Specific Integrated Circuit(s) (ASICs), Field-Programmable Gate Array (FPGAs), electronic circuit(s), central processing unit(s), microprocessor(s), etc. Non-transitory components of the memory 129 used herein are capable of storing machine-readable instructions in the form of one or more software or firmware programs or routines, signal conditioning and buffer circuitry, logic circuit(s), input/output (I/O) circuit(s) and devices, signal conditioning and buffer circuitry, and other components that can be accessed by the processor 29 to provide the described visualization and convergence functionality.

In general, an individual's cognitive system receives several different inputs when creating a 3D perception. Such inputs include motion parallax, depth from motion, perspective, accommodation, occultation, stereopsis, and convergence, among others. The present solutions pertain to the latter, i.e., convergence, which occurs when the eyes of the clinician 22 attempt to focus on a target object arranged at a distance. The angle at which the eyes converge, i.e., the convergence angle, is relatively small when viewing far away objects. VAC-related physiological discomfort is sometimes experienced with stereoscopic displays such as those contemplated herein, as the clinician 22 is required to overcome normal coupling between vergence and accommodation. As a result of implementing the present teachings, short term discomfort (such as the inability to perceive a 3D image) is minimized, as is long term discomfort (e.g., minutes to hours) in the form of fatigue, headache, or nausea.

Referring now to FIG. 2, the digital binoculars 16 include the left front lens 320L and the right front lens 320R, which are arranged on a horizontal plane 40 such that the lenses 320L and 320R are equidistant from a left and right eye 22EL, 22ER of the clinician 22 illustrated in FIG. 1, thus forming a stereoscopic system. The left and right front lenses 320L and 320R are arranged on respective first and second optical axes (AA-L and AA-R). As noted above, first and second micro-displays 25L and 25R are horizontally decentered such that a first and second center axis (BB-L and BB-R) of the respective micro-displays 25L and 25R are not coaxially aligned with a corresponding optical axes (AA-L or AA-R). A convergence angle (γ) is thus formed between the left and right front lenses 320L and 320R and a target object 50 (“Perceived Image”), which in this exemplary use case is a projected image of a patient's eye.

The left front lens 320L shown in FIG. 2 acts as a first front lens having the first optical axis (AA-L) in this embodiment. The second (right front) lens 320R has the second optical axis (AA-R), which in turn is parallel to the first optical axis (AA-L). The first micro-display 25L is positioned within the housing cavity 240 and configured to present a first image from one of the image sensors 200 (FIG. 1), with the first micro-display 25L having the first center axis (BB-L) arranged at a predetermined angle with respect to the first optical axis (AA-L). Although the actual angle may vary with the application, representative predetermined angles of less than about 5° are contemplated herein as set forth below.

The second micro-display 25R in this embodiment is positioned within the housing cavity 240 of FIG. 1 and configured to present a second image from another of the image sensors 200 of FIG. 1. The second micro-display 25R includes the second center axis (BB-R), which is arranged at the predetermined angle with respect to the second optical axis (AA-R) such that the first micro-display 25L and the second micro-display 25R are horizontally decentered relative to the first optical axis (AA-L) and the second optical axis (AA-R), respectively.

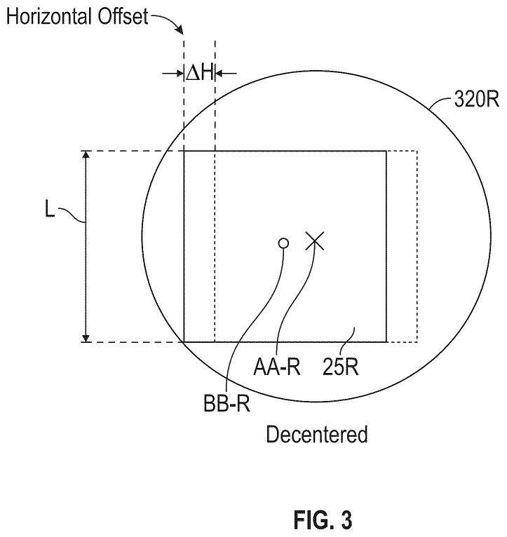

Referring to FIG. 3, each micro-display 25 of FIG. 1 is constructed herein as a miniature television screen, e.g., a light-emitting diode (LED) screens or displays, organic light-emitting diode (OLED) screens/displays, or liquid crystal on silicon screen. A projected image on a given micro-display 25 of FIG. 1 is ultimately magnified or reduced in size by the lenses 320L, 320R of FIG. 2, with the right lens 320R and second (right) micro-display 25R shown in FIG. 3 for illustrative clarity and simplicity. Embodiments of the first and second micro-displays 25L and 25R may have a square perimeter shape, in which case the micro-displays 25R, 25L would have equal side lengths (L). In a non-limiting/exemplary embodiment, the side length (L) is about 20 mm to about 25 mm, with 21 mm being a typical commercially-available OLED construction.

Also illustrated in FIG. 3 is the above-noted second optical axis (AA-R) of the second/right lens 320R and the second center axis (BB-R) of the second micro-display 25R. By horizontally offsetting the second micro-display 25R a short distance from the optical axis (AA-R), a desirable amount of convergence is attained. For instance, for a representative second micro-display 25R having a side length (L) of 21 mm, the horizontal offset (ΔH) may be about 0.5 mm to about 1.5 mm in a possible implementation, with the first micro-display 25L of FIG. 2 being configured in the same manner.

Decentering as contemplated herein is not limited to optical decentering. Offsetting the second micro-display 25R of FIG. 3 from the second optical axis (AA-R) by a fixed distance can result in some amount of distortion on the corners of the projected image 50 (FIG. 2). Therefore, fixed optical decentering may be combined in one or more embodiments with digital decentering to provide the desired convergence effects.

As appreciated in the art, one may digitally manipulate an image to move the image off center and change its aspect ratio. Doing so in the present application does not affect image resolution, as the digital camera 20 of FIG. 1 may have an exemplary resolution of 1080p (1920 pixels×1080 pixels) compared to the resolution of the micro-displays 25, e.g., 2560×2560. A 1 mm offset would be about 5% of the 21 mm side length (L), or about 122 pixels out of an available 2560. Thus, digital decentering may be added in some implementations to fine tune the convergence results.

For instance, one may use a smaller optical offset of 0.5 mm and a variable digital offset of, e.g., 0-1 mm, to provide the desired convergence effect. In a possible implementation, the processor 29 of FIG. 1 may be configured to digitally decenter images on the first micro-display 25L and the second micro-display 25R of FIG. 2. To that end, the user interface device 30 of FIG. 1 may be used to generate the control signal (CC₃₀) to the processor 29, with the processor 29 configured to digitally decenter the images on the first micro-display 25L and the second micro-display 25R by a variable distance of up to about 1 mm in response to the control signal (CC₃₀) in some implementations. Although shown schematically in FIG. 1 for illustrative simplicity, the user interface device 30 may include, e.g., a touch screen, voice command module, foot-operated or hand-operated pedal or other analog device, etc., enabling the clinician 22 of FIG. 1 to request a particular amount of digital decentering as desired.

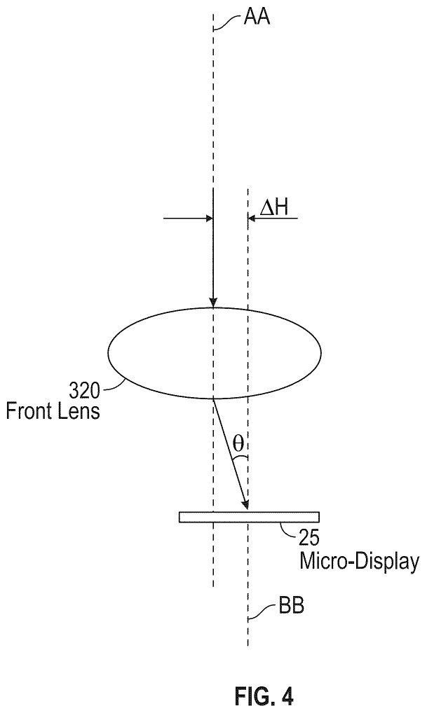

Referring briefly to FIG. 4, which shows a generic micro-display 25 and front lens 320 without regard to its left or right position, and keeping with the nominal 1 mm horizontal offset example introduced above, the type of eyepiece 32/front lens 320 may affect the partial/single-lens angle (θ) between the optical axis (AA) and the center axis (BB) of the micro-display 25. For instance, a horizontal offset (ΔH) of 1 mm for one type of front lens 320 may result in a partial/single-lens angle of 1.7°, or a convergence angle (γ) of 3.4°. The same horizontal offset (ΔH) used with another type of lens 320 could result in a partial/single-lens angle (θ) of about 2.1°, or a total convergence angle (γ) of 4.2°. Thus, the actual optical and possible digital decentering used in a particular implementation will be expected to vary with the type of lens 320 and the design of the digital binoculars 16, including an interpupillary distance between the eyepieces 32 of FIG. 1.

Embodiments shown in the drawings or the characteristics of various embodiments mentioned in the present description are not necessarily to be understood as being independent of each other. It is possible that each of the characteristics described in a given embodiment could be combined with one or more other desired characteristics from other embodiments, resulting in other embodiments not described in words or by reference to the drawings.

As those of ordinary skill in the art will understand, various features illustrated and described with reference to any one of the figures can be combined with features illustrated in one or more other figures to produce embodiments that are not explicitly illustrated or described. The combinations of features illustrated provide representative embodiments for typical applications. Various combinations and modifications of the features consistent with the teachings of this disclosure, however, could be desired for particular applications or implementations.

Certain terminology may be used in the following description for the purpose of reference only, and thus are not intended to be limiting. For example, terms such as “above” and “below” refer to directions in the drawings to which reference is made. Terms such as “front,” “back,” “fore,” “aft,” “left,” “right,” “rear,” and “side” describe the orientation and/or location of portions of the components or elements within a consistent but arbitrary frame of reference which is made clear by reference to the text and the associated drawings describing the components or elements under discussion. Moreover, terms such as “first,” “second,” “third,” and so on may be used to describe separate components. Such terminology may include the words specifically mentioned above, derivatives thereof, and words of similar import.

For purposes of this disclosure, unless specifically disclaimed, the singular includes the plural and vice versa (e.g., indefinite articles “a” and “an” should generally be construed as meaning “one or more”); the words “and” and “or” shall be both conjunctive and disjunctive; the words “any” and “all” shall both mean “any and all”; and the words “including,” “containing,” “comprising,” “having,” and the like, shall each mean “including without limitation.” Moreover, words of approximation, such as “about,” “almost,” “substantially,” “generally,” “approximately,” and the like, may each be used herein to denote “at, near, or nearly at,” or “within 0-5% of,” or “within acceptable manufacturing tolerances,” or any logical combination thereof.

Accordingly, such other embodiments fall within the framework of the scope of the appended claims. The detailed description and the drawings are supportive and descriptive of the disclosure, but the scope of the disclosure is defined solely by the claims. While various modes for carrying out the claimed disclosure have been described in detail, various alternative designs and embodiments exist for practicing the disclosure defined in the appended claims.

We claim:

1. A digital ocular system for use with image sensors and an ophthalmic microscope, comprising:
a microscope housing configured to connect to the ophthalmic microscope and defining a housing cavity;
a first front lens having a first optical axis;
a second front lens having a second optical axis that is parallel to the first optical axis;
a first micro-display positioned within the housing cavity and configured to present a first image from the image sensors, the first micro-display having a first center axis arranged at a predetermined angle with respect to the first optical axis; and
a second micro-display positioned within the housing cavity and configured to present a second image from the image sensors, the second micro-display having a second center axis arranged at the predetermined angle with respect to the second optical axis, such that the first micro-display and the second micro-display are horizontally decentered relative to the first optical axis and the second optical axis, respectively.

2. The digital ocular system of claim 1, wherein the first micro-display and the second micro-display are square and have equal side lengths of about 20 mm to about 25 mm.

3. The digital ocular system of claim 2, wherein the first center axis and the second center axis are respectively offset from the first optical axis and the second optical axis by at least about 0.5 mm.

4. The digital ocular system of claim 3, wherein the first center axis and the second center axis are respectively offset from the first optical axis and the second optical axis by 0.5 mm to 1.5 mm.

5. The digital ocular system of claim 1, wherein the first micro-display and the second micro-display are light-emitting diode or organic light-emitting diode displays.

6. The digital ocular system of claim 1, wherein the predetermined angle is less than about 2° such that the digital ocular system provides a convergence angle of less than about 4°.

7. The digital ocular system of claim 1, further comprising:
a processor, wherein the processor is configured to digitally decenter images on the first micro-display and the second micro-display.

8. The digital ocular system of claim 7, further comprising:
an interface device, wherein the processor is configured to digitally decenter the images on the first micro-display and the second micro-display by a variable distance of up to about 1mm in response to a control signal from the interface device.

9. A visualization system, comprising:
an ophthalmic microscope;
a pair of image sensors connected to the microscope; and
a digital ocular system having:
a binocular housing configured to connect to the ophthalmic microscope and defining a housing cavity;
a first front lens having a first optical axis;
a second front lens having a second optical axis that is parallel to the first optical axis;
a first micro-display positioned within the housing cavity and configured
to present a first image from the image sensors, the first micro-display having a first center axis arranged at a predetermined angle with respect to the first optical axis; and
a second micro-display positioned within the housing cavity and configured to present a second image from the image sensors, the second micro-display having a second center axis arranged at the predetermined angle with respect to the second optical axis, such that the first micro-display and the second micro-display are horizontally decentered relative to the first optical axis and the second optical axis, respectively.

10. The visualization system of claim 9, wherein the ophthalmic microscope is a digital or hybrid ophthalmic microscope.

11. The visualization system of claim 9, wherein the first micro-display and the second micro-display have a square perimeter with a side length of about 20 mm to about 25 mm.

12. The visualization system of claim 11, wherein the first center axis and the second center axis are respectively offset from the first optical axis and the second optical axis by at least about 0.5 mm.

13. The visualization system of claim 12, wherein the first center axis and the second center axis are respectively offset from the first optical axis and the second optical axis by 0.5 mm to 1.5 mm.

14. The visualization system of claim 9, wherein the first micro-display and the second micro-display are light-emitting diode or organic light-emitting diode displays.

15. The visualization system of claim 9, wherein the predetermined angle is less than about 2° such that the digital ocular system provides a convergence angle of less than about 4°.

16. The visualization system of claim 9, further comprising:
a processor configured to digitally decenter images on the first micro-display and the second micro-display in response to a control signal.

17. The visualization system of claim 16, further comprising:
an interface device configured to output the control signal, wherein the processor is configured to digitally decenter the images on the first micro-display and the second micro-display by a variable distance of up to about 1 mm in response to the control signal from the interface device.

18. The visualization system of claim 9, wherein the image sensors include complementary metal-oxide-semiconductor (CMOS) sensors or charge-coupled device (CCD) sensors.

19. A digital ocular system for use with image sensors and an ophthalmic microscope, comprising:
a microscope housing configured to connect to the ophthalmic microscope and defining a housing cavity;
a first front lens having a first optical axis;
a second front lens having a second optical axis that is parallel to the first optical axis;
a first micro-display positioned within the housing cavity, having a side length of about 20 mm-25 mm, and configured to present a first image from the image sensors, the first micro-display having a first center axis arranged at an angle of less than about 2° with respect to the first optical axis; and
a second micro-display positioned within the housing cavity, having a side length of about 20 mm-25 mm, and configured to present a second image from the image sensors, the second micro-display having a second center axis arranged at the angle of less than about 2° with respect to the second optical axis, such that the first micro-display and the second micro-display are horizontally decentered relative to the first optical axis and the second optical axis, respectively, by about 0.5 mm to about 1.5 mm.

20. The digital ocular system of claim 19, further comprising:
a processor; and
an interface device, wherein the processor is configured to digitally decenter the first image on the first micro-display and the second image on the second micro-display by a variable distance in response to a control signal from the interface device.
